(19) United States
(12) Patent Application Publication
(10) Pub. No.: US 2007/0142716 A1
(43) Pub. Date: Jun. 21, 2007

(54) System and method for generating a patient clinical status indicator

(75) Inventor: James W. Biondi, North Haven, CT (US)

Correspondence Address: Kirkpatrick & Lockhart Preston Gates Ellis LLP, STATE STREET FINANCIAL CENTER, One Lincoln Street, BOSTON, MA, 02111-2950, US

(73) Assignee: Cardiopulmonary Corporation, Milford, CT (US)

(21) Appl. No.: 11/640,452

(22) Filed: Dec. 15, 2006

Related U.S. Application Data
(60) Provisional application No. 60/750,533, filed on Dec. 15, 2005.

(51) Int. Cl. A61B 5/00; A61B 5/02; A61B 5/08

(52) U.S. Cl. 600/301; 600/300; 600/485; 600/500; 600/529; 600/549

(57) ABSTRACT

A system and method for displaying a patient clinical status indicator. In one embodiment the system includes a plurality of sensors, each sensor measuring a respective patient parameter; a processor in communication with each of the plurality of sensors, and a display in communication with the processor. The processor receives the patient parameters and generates a patient clinical status indicator in response to the plurality of patient parameters. The display displays the patient clinical status indicator. In one embodiment the patient clinical status indicator is a risk indicator. In one embodiment, the method includes the steps of measuring a plurality of patient parameters; generating a patient clinical status indicator in response to the plurality of patient parameters; and displaying the patient clinical status indicator. In another embodiment the step of generating the patient clinical status indicator further uses a plurality of weighing coefficients each associated with a respective patient parameter.

### FIELD OF THE INVENTION

The invention relates generally to the field of patient monitoring and more specifically to the field of data analysis used in patient monitoring.

This application claims priority to U.S. Provisional Patent Application 60/750,533 filed on Dec. 15, 2005, the disclosure of which is herein incorporated by reference in its entirety.

There are a multitude of patient parameters available to the clinician or care provider for monitoring. Many of the parameters comprise real-time physiologic monitoring of the patient. As a result, especially in critical care environments, the amount of data, much of which is time-sensitive, presented to the caregiver is voluminous and as a result the caregiver may not notice trends, or changes in the patient's parameters in a timely, clinically relevant manner. The present invention provides a solution to this problem.

### SUMMARY OF THE INVENTION

The invention relates in one aspect to a system for displaying a patient clinical status, alert and/or alarm indicator. In one embodiment, the system includes a plurality of sensors, each sensor measuring a respective patient clinical or physiologic parameter; a processor in communication with each of the plurality of sensors, and a display in communication with the processor. The processor receives the patient parameters and generates a patient clinical status indicator in response to the plurality of patient parameters. The display displays the patient clinical status indicator generated by the processor.

In one embodiment, the patient clinical status indicator is a risk indicator. In another embodiment the patient clinical status indicator is a predictive outcomes indicator. In still another embodiment, the system communicates medical device alerts and alarms to the display. In yet another embodiment the patient clinical status indicator is generated in response to a plurality of weighing coefficients, each associated with a patient parameter. In one embodiment the plurality of patient parameters include temperature, blood pressure, respiration rate, blood oxygen, respiration volume, and pulse rate. In another embodiment the patient clinical status indicator is calculated from a polynomial which includes one or more of the plurality of patient parameters and a plurality of coefficients. Each of the coefficients is associated with a respective parameter.

In another aspect, the invention relates to a method for displaying a patient clinical status indicator. In one embodiment, the method includes the steps of measuring a plurality of patient parameters; generating a patient clinical status indicator in response to the plurality of patient parameters; and displaying the patient clinical status indicator. In another embodiment the step of generating the patient clinical status indicator further uses a plurality of weighing coefficients each associated with a respective patient parameter. In another embodiment, the plurality of patient parameters include temperature, blood pressure, respiration rate, respiration volume, and pulse rate, etc. In yet another embodiment, the step of generating the patient clinical status indicator includes calculating a polynomial comprising the plurality of patient parameters and a plurality of coefficients, each coefficient associated with a respective patient parameter.

### BRIEF DESCRIPTION OF THE DRAWINGS

The foregoing and other objects, aspects, features, and advantages of the invention will become more apparent and may be better understood by referring to the following description taken in conjunction with the accompanying drawings, in which:

FIG. 1 is a block diagram of an illustrative embodiment of a system constructed in accordance with the invention; and

FIG. 2 is a flow chart of an illustrative embodiment of the method performed in accordance with the invention.

### DETAILED DESCRIPTION

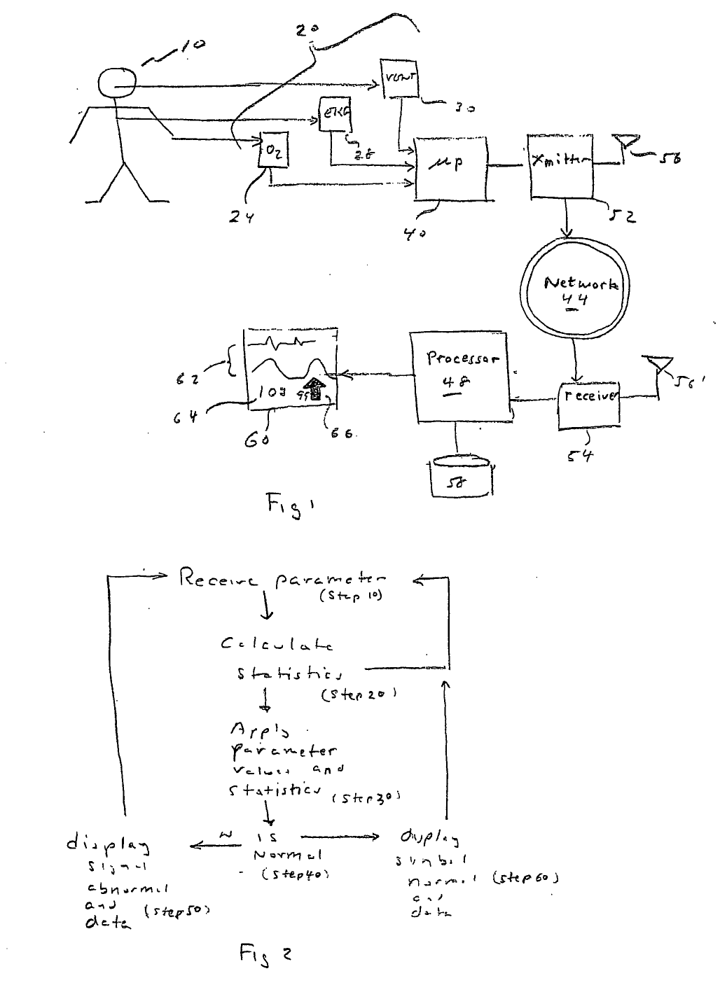

In brief overview and referring to FIG. 1, a patient 10 is monitored by a number of physiologic patient monitors, generally 20. These monitors can include one or more of oxygen sensors 24, carbon dioxide and other metabolic sensors, electrocardiogram (cardiac) 28, hemodynamic (e.g., blood pressure, pulse pressure, blood volume, and blood flow) monitors and ventilation/respiratory monitor 30. Each of these monitors 20 produces one or more signals which are input signals to a processor 40. These signals are processed and either transmitted over a network 44 to a host processor 48 or communicated to a transmitter 52 which broadcasts the signals to a receiver 54 by way of antennae 56, 56′. In one embodiment the network is hard-wired rather than wireless. The host processor 48 performs calculations on the signals and then transmits the results to a display 60. The display 60 displays the results of the calculations including the original signals 62, a numerical score 64 and a trend indicator 66. In another embodiment alarms and alerts from the monitors are communicated directly to the display. In other embodiments status change and normal limit indicators are also displayed. In other embodiments alarms are generated when trends are detected that are detrimental to the patient.

In more detail, the patient 10 has attached a number of sensors each of which is connected to a specific monitor 20. A typical patient 10 might be monitored by an oxygen sensor 24 attached to the patient's finger, airway respiratory gas sensors and detectors, a plurality of electrocardiographic electrodes attached to an EKG monitor 28, hemodynamic sensors (including, but not limited to, blood pressure, pulse pressure, blood flow and blood volume) and a respiratory monitor 30. Each of the monitors 20 produces one or more output signals in response to the input signals provided by the sensors. For example the oxygen monitor 24 may produce a single value, oxygen concentration in the blood, while the EKG monitor 28 may produce multiple signals, including heart rate and ecg waveforms. Hemodynamic sensors may monitor such parameters as blood pressure, blood flow, blood volume and cardiac output. Body surface sensors and implanted biosensors measure various physiologic functions which are also monitored.

Further, the output signals from the monitors 20 may be substantially the same as received from the sensors or processed. As a result, the signals which are input signals to the processor 40 may be an analog or digital form. If they are in analog form, the input signals are first processed by an analog to digital converter (A/D) before being sent for processing by the processor 48. If the signals are pre-processed by the monitor 20 and have a digital format, input to the processor 40 can be through a serial or parallel digital input device.

The processor 40 then packages the data from the monitors 20 for communication to a transmitter 52. The packaging of the data includes in one embodiment inserting a patient ID number with the data. In addition to packaging the various data for transmission, the processor 40 may also encrypt the data. The transmitter 52, in one embodiment, is a modem to connect the processor 40 to a wired network 44. The network can be a local area or wide area network. In a second embodiment the transmitter 52 is a WIFI, (or other wireless band) transmitter that transmits the data by way of an antenna 56 over a wireless network. In a third embodiment the transmitter is a transceiver for transmission of data over a hard-wired network such as RS 232 or ethernet.

The data is received from the network 44 or the WIFI network, through receiver antenna 56′, by a receiver 54 that provides the data to a host processor 48. In another embodiment the receiver is a transceiver which receives data over a hand-wired network such as an RS 232 or ethernet network. The host processor 48, uses the data for statistical analysis, writes the data to a database 58 and applies rules to the statistically processed data or unprocessed data as described below. The host processor 48 then prepares the data for display on a monitor 60.

The displayed data typically includes the data waveforms 62, but the processed numerical data 64 and calculated values and indicia of status 66. These calculated values include risk indicators and predictive outcome indicators as described below.

In various embodiments alarms/alerts generated within the monitors are communicated for immediate display. In other embodiments alarms arising from calculations based on the parameters received from the monitors, such as trend, status change and normal limit indicators are displayed.

In operation, referring to FIG. 2, the system receives (step 10) a parameter value from a monitor on a given patient. This data is added to a database and used to calculate statistics (step 20) regarding the behavior of the parameter. This calculation loop is continued for a predetermined number of cycles. With the parameter statistics gathered, the parameter and the statistics are applied to a rule set (step 30) to determine if the parameter, statistics and parameter trend are indicative of a change in the status of the patient (step 40) as described below. If the results are normal, the data, risk value and status indicator, such as an upwardly pointing arrow are displayed (step 60). If the results are abnormal, the data, risk value and indicator; such as a downwardly pointing arrow are displayed (step 50). As described, the system may automatically triage patients based on clinical status and relative risk. The system, in other embodiments, provides other means of decision support to control therapy. This clinical decision support may use rules that may incorporate on evidence based outcome indicators to tailor treatment, based on, for example, patient trends. That is, for example, if a patient's blood pressure is decreasing, the infusion of blood pressure drugs can be decreased or stopped. Ventilation parameters may be changed to help support the patient's O₂ trend.

To determine if the patient parameters indicate that the patient is in increasing or decreasing risk, several calculations may be performed. First, a polynomial may be generated which takes into account the parameters of interest, defines their importance by the power of the variable to which the parameter corresponds, and applies a weighting coefficient to each parameter to rank parameters of the same power relative to one another. So for example an equation in one embodiment is as follows:
Risk index=(A(Hrate−Hrate baseline)^{L}+B(Hirregularity)^{M}+C(O₂—O_{2 ave})^{N}+D(Hrate−Hrate max)^{O}+E(T-Tnormal)^{P}+F(bp−bpbaseline)^{Q}+G(Systolic−Diastolic Pressure)^{R}+H(Pulse Pressure)^{S}+I(Cardiac Output)^{T}+J(Flow/time)^{U}+K(Other Parameter)^{V}

In this equation, deviation from the baseline heart rate (Hrate−Hrate baseline); heartbeat irregularity (Hirregularity); and deviation of blood oxygen concentration from average blood oxygen concentration (O₂—O_{2 min}) are important, but only as a linear function of their deviation. Their relative importance is determined by the values of the coefficients A, B, C through the last parameter coefficient (K). Exponents L, M, N through the last parameter exponent (V) determine the relative importance of the parameter or its deviation from some set valve. For example, deviation from the maximum acceptable heart rate limit (Hrate−Hrate max) and deviation from normal temperature (T-Tnormal) may be more significant and as a result a heart rate greater than the maximum allowable may be raised to the second power and deviation in temperature may be cubed. Thus, in this case deviation from normal temperature and heat rate will have a greater effect on the risk index than a change in O₂ concentration. The coefficients A, B, C are used to weigh the relative importance of the variables which are of the same power. The coefficients may be chosen as a normalizing number make the risk index fall between some values, for example 1 and 100.

In another embodiment, the various parameters are subjected to a multivariate analysis.

The methods and systems described herein can be performed in software on general purpose computers, servers, or other processors, with appropriate magnetic, optical or other storage that is part of the computer or server or connected thereto, such as with a bus. The processes can also be carried out in whole or in part in a combination of hardware and software, such as with application specific integrated circuits. The software can be stored in one or more computers, servers, or other appropriate devices, and can also be kept on a removable storage media, such as a magnetic or optical disks. Furthermore, the methods described herein can be implemented using as an SDK, an API, as middleware, and combinations thereof.

The foregoing description has been limited to a few specific embodiments of the invention. It will be apparent, however, that variations and modifications can be made to the invention, with the attainment of some or all of the advantages of the invention. It is therefore the intent of the inventors to be limited only by the scope of the appended claims.

What I claim is:

1. A system for displaying a patient clinical status indicator comprising:
a plurality of sensors, each sensor measuring a respective patient parameter;
a processor in communication with each of the plurality of sensors, the processor receiving the patient parameters and generating a patient clinical status indicator in response to the plurality of patient parameters; and
a display in communication with the processor, the display displaying the patient clinical status indicator.

2. The system of claim 1 wherein the patient clinical status indicator is a risk indicator.

3. The system of claim 1 wherein the patient clinical status indicator is a predictive outcomes indicator.

4. The system of claim 1 wherein the patient clinical status indicator is generated in response to a plurality of weighing coefficients each associated with a patient parameter.

5. The system of claim 1 wherein the plurality of patient parameters comprise temperature, blood pressure, pulse rate, respiration rate, blood oxygen level, respiration tidal volume, and, expired respiratory gas, urine output, clinical blood chemistries, or other clinical signs or physiologic parameters.

6. The system of claim 1 wherein the patient clinical status indicator is calculated from a polynomial comprising the plurality of patient parameters and a plurality of coefficients, each of the coefficients associated with a respective parameter.

7. The system of claim 1 wherein the patient clinical status indicator is calculated from a plurality of parameters in a multivariate, or other mathematic algorithimic analysis.

8. A method for displaying a patient clinical status indicator, the method comprising the steps of:
measuring a plurality of patient parameters;
generating a patient clinical status indicator in response to the plurality of patient parameters; and
displaying the patient clinical status indicator.

9. The method of claim 8 wherein the step of generating the patient clinical status indicator further uses a plurality of weighing coefficients each associated with a respective patient parameter.

10. The method of claim 8 wherein the plurality of patient parameters comprise temperature, blood pressure, respiration rate, respiration volume, and pulse rate.

11. The method of claim 8 wherein the step of generating the patient clinical status indicator comprises calculating a polynomial comprising the plurality of patient parameters and a plurality of coefficients, each associated with a respective patient parameter.

12. The method of claim 8 wherein the step of generating the patient clinical status indicator comprises a multivariate analysis of the parameters.

13. A system for displaying a patient clinical status indicator comprising:
a sensor for monitoring a patient parameter and generating an alarm if the parameter falls outside an acceptable range; and
a display in communication with the sensor over a network, the display displaying the alarm.

14. The system of claim 13 further comprising a processor in communication with the sensor.

15. The system of claim 14, wherein the processor is adapted to provide real-time decision support based on received parameter values.

16. The system of claim 14, wherein the processor is adapted to provide real-time decision support based on changes in parameter values.

17. The system of claim 14, wherein the processor is adapted to provide real-time decision support based on alarm signals.
